(19) United States
(12) Patent Application Publication
(10) Pub. No.: US 2026/0130767 A1
(43) Pub. Date: May 14, 2026

(54) FULLY COMPUTER-INTELLIGENT METHOD AND SYSTEM FOR PRODUCING HIGHLY SIMILAR VACATION BODIES

(71) Applicant: Yulin Lanzhihuixin Medical Technology Co., Ltd., Yulin (CN)

(72) Inventors: Koichi Shimizu, Yulin (CN); Shiyang Liang, Yulin (CN); Jianan Li, Yulin (CN)

(21) Appl. No.: 19/383,516

(22) Filed: Nov. 7, 2025

(30) Foreign Application Priority Data
Nov. 8, 2024 (CN) 202411593191.7

(51) Int. Cl. A61F 2/30

(52) U.S. Cl. CPC A61F 2/30942; A61F 2002/30003; A61F 2002/30985

(57) ABSTRACT

The present invention discloses a fully computer-intelligent method and system for producing high-fidelity prostheses, comprising: a host computer that acquires patient information and utilizes a pre-built artificial intelligence model to generate a 3D model containing segmentation information of tissues within the patient's missing area; based on patient information and the 3D model, it derives a 2D unfolded image corresponding to the surface layer of the 3D model; Controlling a 3D printing device to print layered sections of the prosthesis body from the 3D model; controlling a membrane fabrication device to produce a surface membrane for the missing area; controlling a 2D printing device to print the 2D unfolded image onto the surface membrane of the missing area, forming a skin layer; controlling an assembly device to combine all prosthesis components, affix the skin layer to the prosthesis body surface, and perform surface treatment to produce the finished prosthesis.

### CROSS-REFERENCE TO RELATED APPLICATION

This application claims priority of Chinese Patent Application No. 202411593191.7, filed on Nov. 8, 2024, the entire contents of which are incorporated herein by reference.

### TECHNICAL FIELD

The present invention relates to the field of prosthesis manufacturing, specifically to a fully computer-intelligent high-similarity prosthesis production method and system.

### BACKGROUND TECHNOLOGY

For disabled patients, a well-designed prosthesis not only enables users to perform daily activities independently and comfortably but also helps them build confidence and reintegrate into society, contributing to social stability and development. Traditional prosthetic production methods generally involve the following discrete processes: (1) 3D measurement sampling of the human body shape; (2) 3D printing of the negative mold; (3) Fabrication of the positive mold using body-simulating plastic (e.g., silicone rubber); (4) Manual surface coating; (5) Surface protective treatment.

Although sampling steps in current prosthetic production can be completed using digital instruments, the manufacturing process still relies heavily on manual labor. Consequently, the entire process is not only time-consuming but also demands extensive personal experience and skill from each operator. Surface painting, in particular, requires exceptional technical proficiency and artistic talent, akin to creating body art. Furthermore, when addressing extensive defects or bilateral organ loss, craftsmen must rely on experience gained through years of practice to conceptualize the shape of the missing parts. Since it is difficult to visualize the specific form of the missing body parts, reconstructing asymmetrical areas like lips or noses is challenging when relying solely on manual methods.

### INVENTION SUMMARY

To address the aforementioned issues in existing techniques, the present invention provides a fully computer-intelligent method and system for producing highly similar prostheses. The technical problems addressed by the invention are solved through the following technical solutions:

First Aspect The present invention provides a fully computer-intelligent method for producing highly similar prostheses, comprising:
A main computer collects patient information and utilizes a pre-built artificial intelligence model to generate a 3D model containing segmentation information of various tissues within the patient's missing area. Based on the collected patient information and the 3D model, a 2D unfolded image of the 3D model's surface layer is obtained. The tissues include bone, muscle, tendon, fat layer, and skin layer.
The main computer controls a 3D printing device to print the 3D model, producing the prosthesis body;
The main computer controls the membrane fabrication device to produce a membrane for the surface layer of the missing area;
The main computer controls the 2D printing device to print the 2D unfolded image onto the membrane for the surface layer of the missing area, forming a skin layer;
The main computer controls the assembly device to bond the skin layer onto the surface of the prosthesis body, and after surface treatment, the prepared prosthesis is obtained.

In one embodiment of the present invention, when the healthy reference side lacks symmetry at the missing site, the main computer acquires patient information and utilizes a pre-built artificial intelligence model to obtain a 3D model containing segmentation information of various tissues within the patient's missing site. Based on the acquired patient information and said 3D model, it generates a 2D unfolded image of the 3D model's surface layer, comprising:
The main computer utilizes a pre-built first model reconstruction neural network to perform three-dimensional reconstruction of the missing region by collecting information from the patient's remaining areas, thereby obtaining a reconstructed three-dimensional structural model and a surface image of the reconstructed missing region; wherein the first model reconstruction neural network is implemented using generative AI technology;
Performing structural segmentation of internal tissues on the 3D structural model using a pre-built second structural hierarchical neural network, and modifying the model using model attribute information from the collected patient data and a pre-built third model modification neural network, resulting in a processed 3D model;
Perform a two-dimensional image unfolding on the reconstructed surface image of the missing region based on the 3D model to obtain a 2D unfolded image.

In one embodiment of the present invention, when a symmetrical healthy reference side exists for the missing region, the main computer acquires patient information and utilizes a pre-built artificial intelligence model to obtain a 3D model containing segmentation information of each tissue within the patient's missing region. Based on the acquired patient information and the 3D model, a 2D unfolded image corresponding to the surface layer of the 3D model is obtained, including:
The main computer acquires depth information of the healthy reference side and the remaining portion of the missing area using a depth information acquisition device, and acquires multiple surface images of the healthy reference side at different angles using an image acquisition device;
Performing three-dimensional reconstruction of the missing area using the collected depth information to obtain a reconstructed three-dimensional structural model;
Performing structural segmentation of internal tissues on the three-dimensional structural model using a pre-built second structural hierarchical neural network, and modifying the model using model attribute-related information from the collected patient information and a pre-built third model modification neural network, resulting in a processed 3D model;
Perform two-dimensional image unfolding on the acquired multiple surface images based on the 3D model to obtain 2D unfolded images.

In one embodiment of the present invention, the model attribute-related information includes: the patient's age, gender, height, weight, and skin tone; the model modification includes modifying the prosthesis size, shape, surface color, and surface texture.

In one embodiment of the present invention, the process of obtaining the 2D unfolded image employs a predetermined inverse filtering algorithm. By comparing the digital image of the surface layer of the 3D model with its printed image obtained using a 2D printing device, the algorithm compensates for the visual discrepancies between the digital image and its printed counterpart, thereby generating the 2D unfolded image ready for printing, wherein the digital image comprises: when the missing part lacks a symmetrical healthy reference side, an image obtained by 2D unfolding the surface image reconstructed from the 3D model for the missing part; or, when the missing part has a symmetrical healthy reference side, an image obtained by 2D unfolding multiple collected surface images based on the 3D model.

In one embodiment of the present invention, the process of the predetermined inverse filtering algorithm comprises:
Step c1: Reading initial parameters; wherein the initial parameters include image matrix size, similarity threshold, and maximum iteration count;
Step c2: Acquire the digital image;
Step c3: Print the digital image using a 2D printing device to obtain the original printed image;
Step c4: Acquire images of the digital image and the original printed image using the same image acquisition device employed during the acquisition of the digital image, obtaining corresponding acquired images;
Step c5: Calculate the inverse filtering function using the captured images corresponding to the current digital image and the original printed image;
Step c6: Apply the inverse filtering function to the current digital image to obtain the inverse filtered digital image;
Step c7: Determine whether the current iteration count is less than the maximum iteration count; if yes, proceed to step c8; if no, proceed to step c14;
Step c8: Print the current inverse-filtered digital image using a 2D printing device to obtain the first printed image;
Step c9: Acquire images of the current inverse-filtered digital image and the first printed image using the same image acquisition device, obtaining corresponding acquired images;
Step c10: Calculate the similarity index for the captured images corresponding to the current reverse-filtered digital image and the first printed image;
Step c11: Determine whether the similarity index exceeds the similarity threshold; if yes, proceed to step c12; if no, proceed to step c13;
Step c12: Determine that the currently obtained reverse-filtered digital image serves as the 2D unfolded image to be printed;
Step c13: Store the current reverse-filtered digit image and its similarity index. Replace the current digit image with the current reverse-filtered digit image, replace the current digit image's corresponding captured image with the captured image corresponding to the current reverse-filtered digit image, and replace the current original printed image's corresponding captured image with the captured image corresponding to the current first printed image;
Step c14: Locate the digitally filtered image with the highest similarity index among all iterations and designate it as the 2D unfolded image to be printed.

In one embodiment of the present invention, the main computer controls the 3D printing device to print the 3D model, obtaining the prosthesis body, comprising:

The main computer controls the 3D printing device to print using materials matched to each tissue in the 3D model, obtaining the prosthesis body; wherein the materials matched to each tissue comprise a plurality of optional materials.

In one embodiment of the present invention, for each tissue type, the matched material is determined from multiple optional materials for that tissue based on collected patient information, selecting one that most closely matches the patient's actual human tissue in terms of hardness and viscoelasticity.

In one embodiment of the present invention, the main computer controls the film-making device to manufacture a surface film for the missing area, including:
The main computer controls the film-making device to obtain a sol-state silicone rubber solution, which is placed on printing paper with a preset thickness and surface roughness to form a silicone rubber solution layer of a set thickness;
The main computer-controlled film-making device covers the upper surface of the silicone rubber solution layer with a substrate sheet. After the silicone rubber solution layer gels, it peels off the silicone rubber solution layer to obtain the surface film for the missing area, wherein the original contact surface between the surface film and the printing paper serves as the printing surface; wherein the substrate sheet exhibits higher adhesion strength to the silicone rubber than the printing paper surface, and the printing paper is compatible with the 2D printing device.

In one embodiment of the present invention, the predetermined surface roughness is determined based on the skin surface roughness of the patient obtained from the collected patient information; the predetermined thickness is determined based on the skin thickness of the patient obtained from the collected patient information.

In one embodiment of the present invention, before the main computer controls the 2D printing device to print the 2D unfolded image onto the surface film of the missing area to form the skin covering, the method further comprises:

the main computer controlling the printing system to treat the surface of the surface film of the missing area with a surface treatment solution and uniformly print pigment ink after treatment.

In one embodiment of the present invention, the main computer controls the assembly device to affix the skin covering to the surface of the prosthesis body, including:

The main computer controls the printing system to apply adhesive and lubricant to the surface of the prosthetic body, covers the coated prosthetic body surface with the skin, and controls a uniform pressure instrument to apply pressure, causing the skin and prosthetic body to tightly bond into an integrated structure.

In one embodiment of the present invention, the density and adhesion strength of the adhesive match the collected patient information; The lubricant employs a biocompatible lubricant.

In one embodiment of the present invention, the surface treatment comprises:

The main computer controls the spray coating system to apply a protective layer resembling human skin surface to the surface of the prosthesis body after skin lamination.

Second aspect, the present invention provides a fully computer-intelligent high-similarity prosthesis production system, the system comprising:

A main computer and other components; wherein the main computer controls the other components to perform the method steps described in the first aspect; wherein the other components include an image acquisition and storage system, a film-making device, an assembly device, a printing system, a uniform pressure application instrument, and a spray coating system; wherein the image acquisition and storage system comprises depth information acquisition equipment and image acquisition equipment; wherein the printing system comprises at least 3D printing equipment and 2D printing equipment.

Thirdly, the present invention provides a computer-readable storage medium storing a computer program. When executed by a processor, the computer program implements the steps of the method provided in the first aspect of the present invention.

### Advantages of the Invention

The fully computer-intelligent high-similarity prosthesis production method and system provided by the present invention, controlled by a main computer to complete prosthesis design and production, computerizes and intelligently integrates all processes. This enables the provision of high-precision, high-similarity prostheses in shorter timeframes. By digitizing the production workflow through computer operation, reliance on manual experience or skills is eliminated, significantly reducing labor time and costs.

Furthermore, the present invention incorporates artificial intelligence technology combined with patient information for prosthesis design and modeling. The use of AI not only yields high-precision, high-similarity 3D models containing segmentation information of tissues within the patient's missing area for 3D printing output, enabling segmentation of internal limb structures (such as bones, muscles, tendons, fat layers, skin layers, etc.) and reconstruction of missing structural parts. It also generates highly realistic 2D unfolded images of the 3D model's surface for 2D printing.

Through artificial intelligence algorithms, prosthetic design and production can be completed using the healthy reference side as data when the missing area has a symmetrical healthy counterpart. Furthermore, even when both symmetrical body parts (hands, arms, legs, feet, ears, etc.) are missing, limb restoration can be achieved. Additionally, prosthetic models can be generated for single-site defects (nose, chin, etc.). Compared to traditional methods, this approach enables more effective customization of prostheses for individual patients, achieving high similarity between the prosthesis and the patient's body, thereby enhancing prosthesis quality. It eliminates limitations in prosthesis production for missing body parts, meets the needs of more patients, and improves practicality.

### DESCRIPTION OF THE DRAWINGS

FIG. 1 is a flowchart illustrating the fully computer-intelligent high-similarity prosthetic production method provided by an embodiment of the present invention;

FIG. 2A is a schematic diagram of the training principle of the deep learning neural network model in an embodiment of the present invention;

FIG. 2B illustrates the operational principle of the deep learning neural network model in an embodiment of the present invention;

FIG. 3 depicts the flowchart of the reverse filtering algorithm in an embodiment of the present invention;

FIG. 4 shows the process flowchart for manufacturing the surface membrane of the missing area in an embodiment of the present invention;

FIG. 5 illustrates the working principle of the uniform pressure application device in an embodiment of the present invention;

FIG. 6A illustrates the operational process of the fully computer-intelligent high-similarity prosthesis production system when the missing area has a symmetrical healthy reference side in an embodiment of the present invention;

FIG. 6B illustrates the operational process of the fully computer-intelligent high-similarity prosthesis production system when the missing area lacks a symmetrical healthy reference side in an embodiment of the present invention.

### SPECIFIC IMPLEMENTATION METHODS

The present invention is further described in detail below with reference to specific embodiment, but the implementation methods of the invention are not limited thereto.

To achieve comprehensive computerization and intelligent automation throughout the design and manufacturing process of prostheses, enabling more efficient customization of highly similar prostheses for individual patients compared to traditional methods, the present invention provides a fully computer-intelligent method, system, and computer-readable storage medium for producing highly similar prostheses. This is accomplished through interaction between a main computer and other components, as well as the main computer's control over these components.

The prostheses in the embodiment of the present invention may include various types of artificial limbs and prosthetic devices used in the human body. The body parts involved may include the hand, arm, leg, breast, ear, and so forth.

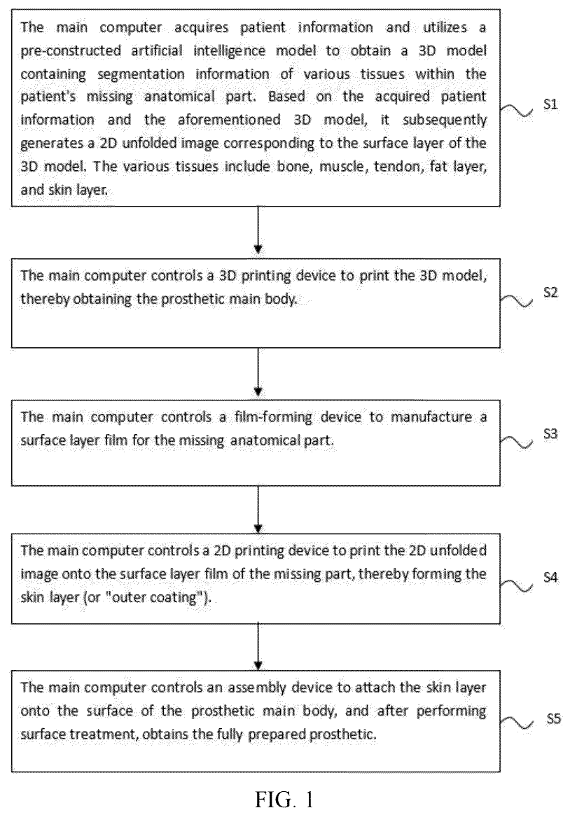

First aspect: The embodiment of the present invention provide a fully computer-intelligent high-similarity prosthesis production method. As shown in FIG. 1, the method may include the following steps:
S1. The main computer acquires patient information and utilizes a pre-built artificial intelligence model to generate a 3D model containing segmentation information of tissues within the patient's missing body part. Based on the acquired patient information and the 3D model, it obtains a 2D unfolded image of the 3D model's surface layer. The tissues include bones, muscles, tendons, fat layers, and skin layers.

The main computer in the embodiment of the present invention may be implemented using any existing computer capable of performing communication, control, and data processing functions.

In the present invention, the main computer can control acquisition devices to collect patient information, such as data on the patient's three-dimensional body structure and skin surface. Utilizing a pre-built artificial intelligence model (where “pre-built” refers to models established and trained beforehand), it generates a 3D model containing segmentation information for each tissue within the patient's missing area. This 3D model can characterize the external shape and internal 3D structural data of the patient's missing area while segmenting and distinguishing internal tissues such as bones, muscles, tendons, fat layers, and skin layers. This facilitates subsequent printing and construction of these distinct internal tissues within the prosthesis body, achieving tissue differentiation inside the printed prosthesis to make it more closely resemble the patient's actual internal structure.

Additionally, based on the acquired patient data and the 3D model, a 2D unfolded image of the model's surface layer can be generated. This 2D unfolded image simulates the actual skin appearance on the surface area of the patient's 3D model. Printing this 2D unfolded image subsequently yields a skin pattern resembling the patient's skin surface.

Specifically, many human body parts exhibit symmetrical distribution, such as arms, legs, and ears, while others lack a symmetrical counterpart on the opposite side, such as the mouth or nose, or both sides may be absent. Therefore, the present invention primarily addresses two scenarios: cases where the missing part lacks a symmetrical healthy reference side, and cases where the missing part has a symmetrical healthy reference side. The following describes the specific implementation process of S1 for these two scenarios.

For the scenario where the missing part lacks a symmetrical healthy reference side, the main computer acquires patient information and utilizes a pre-built artificial intelligence model to obtain a 3D model containing segmentation information of various tissues within the patient's missing part. Based on the acquired patient information and the 3D model, it generates a 2D unfolded image of the 3D model's surface layer, including:
Step a1: The main computer utilizes a pre-built first model reconstruction neural network to perform three-dimensional reconstruction of the missing region by collecting information from the patient's remaining areas. This yields a reconstructed three-dimensional structural model and a reconstructed surface image of the missing region.

For cases where the missing region lacks a symmetrical healthy reference side—such as when symmetrical regions exist but both sides are missing, or no symmetrical regions are present—data from the healthy reference side cannot be directly used as reconstruction reference for the missing side. Therefore, the present invention pre-constructs a first model reconstruction neural network to identify and restore the missing body shape. This neural network collects 3D information from all areas of the patient except the missing region to perform 3D reconstruction of the missing area, yielding a reconstructed 3D structural model. Simultaneously, step a1 acquires surface information from the patient's remaining body parts (excluding the missing area). This information is applied to the reconstructed 3D structural model to generate a surface image of the reconstructed missing area—i.e., an image of the surface of the missing area's 3D structure. For example, if the missing area is the forearm, the reconstructed surface image corresponds to the circumferential surface of the forearm.

The first model reconstruction neural network is implemented using generative AI (Artificial Intelligence) technology.

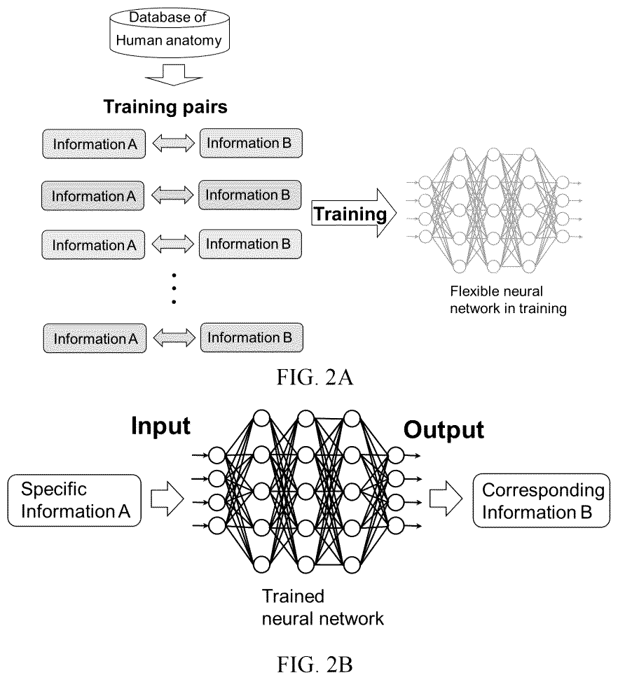

Specifically, the present invention establishes a human anatomy database (or human structure database) containing external morphological data related to age, gender, height, weight, skin tone, etc., along with three-dimensional structural information of the human body. This includes external structural 3D data and internal structural details such as the distribution and positioning of bones, muscles, tendons, fat layers, skin layers, and the viscoelastic properties of various regions. By constructing a deep learning neural network model and training it using a large amount of information A and information B obtained from this database, the trained deep learning neural network model can output corresponding information B based on input information A for specific purposes. The training principle and usage principle of the deep learning neural network model are briefly illustrated in FIG. 2A and FIG. 2B.

Based on the implementation concept of the deep learning neural network model utilizing this human anatomy database, the present invention's embodiment can realize deep learning neural network models for multiple purposes. It should be noted that the training methods for these deep learning neural network models are based on existing neural network training approaches. The focus of the present invention's embodiment lies in constructing corresponding training sets for different purposes—i.e., different input information A and output information B—based on the relevant data in the human anatomy database. These training sets contain training samples corresponding to known input information A, which carry label data corresponding to output information B. This enables the training of deep learning neural network models for specific purposes. Upon completion of training, the model can then perform the process of inputting information A and outputting information B for that specific purpose.

For example, the first model reconstruction neural network represents one type of deep learning neural network model. When utilizing this first model reconstruction neural network, its input information A comprises three-dimensional data of all patient body regions except the missing area. This three-dimensional data may be acquired using a depth information acquisition device.

Such depth information acquisition devices may include X-ray machines, CT (Computed Tomography) devices, MRI (Magnetic Resonance Imaging) devices, ultrasound echo imaging devices, and others. These medical imaging devices can acquire depth information of the human body through scanning or similar methods. From this depth information, the three-dimensional shape of the human body, as well as the structure, composition, and corresponding positional information of its internal parts, can be determined.

To generate the missing portion of the asymmetrical healthy reference side, the present invention divides the entire body shape into distinct segments such as ears, eyes, nose, chin, fingers, palms, forearms, upper arms, breasts, buttocks, upper limbs, lower limbs, and soles of the feet. At this point, information B represents one such segment, while information A can be the integral of all remaining segments. Consequently, by inputting the 3D information of the remaining body parts into a trained deep learning neural network model (i.e., the first model reconstruction neural network), the shape and 3D information of the missing body part can be obtained. This yields a reconstructed 3D structural model of the missing part (encompassing both external shape and internal structure). For example, if a patient is missing their left arm, three-dimensional information from the remaining body parts can be collected and input into the trained first model reconstruction neural network. For the input information A, this first model reconstruction neural network outputs a three-dimensional structural model reconstructing the left arm, serving as the corresponding information B. This reconstructed 3D structural model represents the three-dimensional structure of the left arm. Since the first model reconstruction neural network was trained using a large volume of patient training samples and labeled data, it establishes a feature mapping relationship between input and output. This enables the reconstructed 3D structural model to maximally simulate the actual structure of the missing part belonging to the patient associated with input information A, achieving good matching with the patient's remaining intact parts. This enhances the authenticity and structural similarity of the reconstruction.

The first model reconstruction neural network may adopt existing architectures such as CNN or Point Net. It is understood that for three-dimensional reconstruction of the missing region, the training samples for this first model reconstruction neural network originate from the three-dimensional information of the remaining intact regions of a specific patient within the human anatomy database. This three-dimensional information can undergo certain preprossessing steps, such as dimensionality normalization, filtering, and integration calculations, to obtain the training samples. The corresponding label data for these training samples is the missing region's 3D information—i.e., the 3D structural model. It is understood that constructing the training sample-label data pairs can originate from scenarios where patients have actual missing regions. For example, if a patient genuinely lacks their left hand, the training samples would be the 3D information of all other body parts except the left hand. The label data represents the acquired three-dimensional information of the left hand, i.e., the three-dimensional structural model. This could be three-dimensional information collected and stored before the patient's left hand was lost, three-dimensional information obtained by measuring the patient's left prosthesis, or three-dimensional information of the patient's left hand obtained through other means. Alternatively, training sample-label data can be constructed from intact human data. For instance, anatomical databases store extensive 3D information of intact human body parts. Any specific part—such as the left ear—can be designated as the “missing part.” In this case, 3D information from all other body parts forms the training samples, while the left ear's 3D data serves as the label data. This approach yields numerous training sample-label data combinations tailored to different missing body parts. Therefore, at least through the aforementioned methods of real and artificial missing parts, multiple training sample-label data combinations can be constructed for different missing parts. This forms a training set for training the first model reconstruction neural network. The present invention's embodiment can combine anatomical data from both fully healthy human bodies and human bodies with limb deficiencies to construct this anatomical database, enriching the types of data samples and enhancing the model's robustness.

When training the first model reconstruction neural network using the aforementioned training set, a specific loss function can be set to compare the network's output results with the label data of the training samples. Methods such as backpropagation are then employed to adjust the network parameters. After multiple iterations of training, the loss function gradually converges, meaning the network's output results progressively approach the label data. Ultimately, the first model reconstruction neural network is trained to completion. The specific training process can be understood in conjunction with conventional neural network training procedures.

In this context, the deep learning neural network model of the present invention's embodiments—such as the pre-constructed first model reconstruction neural network—may be deployed either on the host computer or on other devices. For instance, it could reside within a processor, with the host computer invoking the deep learning neural network model via programmatic calls. Both approaches are valid and not restricted herein.

As described above, in step a1, the main computer utilizes the pre-constructed first model reconstruction neural network to perform 3D reconstruction of the missing region by acquiring information from the patient's remaining areas, thereby obtaining a reconstructed 3D structural model. Similarly, the pre-constructed first model reconstruction neural network can also be used to obtain a surface image of the reconstructed missing region.

To obtain the reconstructed surface image of the missing region, after reconstructing the three-dimensional structural model using the pre-built first model, information from the patient's remaining body parts must also be utilized. Here, the information from the patient's remaining body parts refers to the surface images (i.e., body surface information) collected from these areas. This collection process can be performed concurrently with the collection prior to reconstructing the three-dimensional structural model or separately.

The collection of surface images from the patient's remaining areas can be achieved using image acquisition devices, which may include cameras (e.g., digital cameras), video cameras, 3D scanners, etc., without limitation.

To enhance the accuracy of the model output and thereby obtain high-quality reconstructed surface images with high similarity to the patient's body surface, surface images from the patient's remaining areas can be collected from multiple angles to enrich the data volume. Surface image acquisition for the patient's remaining areas also includes cross-sectional images.

The acquired surface images of the patient's remaining areas can undergo certain preprocessing steps. Similar to the 3D reconstruction of missing regions, this preprocessing may involve standardizing image dimensions, applying filters, calculating the integral of all surface images, etc. After preprocessing, these images serve as part of the input information A for the first model reconstruction neural network. At this point, the input information A should also include the three-dimensional information of the reconstructed missing region. After feeding the complete input information A into the first model reconstruction neural network, the reconstructed surface image of the missing region can be obtained, yielding the corresponding output information B.

In other words, in the embodiments of the present invention, the pre-constructed first model reconstruction neural network can first be utilized to perform three-dimensional reconstruction of the missing region based on the collected three-dimensional information from the patient's remaining regions, yielding a reconstructed three-dimensional structural model. Subsequently, based on the reconstructed three-dimensional structural model and the collected surface images from the patient's remaining regions, the first model reconstruction neural network is employed again to obtain the reconstructed surface image of the missing region.

In the above process, the first model reconstruction neural network used in both instances may share the same network architecture but employ distinct network parameters. These parameters are determined and stored through respective training processes. When utilizing this first model reconstruction neural network, a set of adapted network parameters can be invoked based on different input information A to enable the model to recognize and output corresponding information B, thereby achieving the intended purpose. The aforementioned process can be implemented based on existing AI technology. The process of invoking adapted network parameters can be achieved through computer programs, which are not elaborated upon here.

For reconstructing surface images of missing regions, the first model reconstruction neural network is trained using samples sourced from surface images of the patient's remaining intact anatomical regions within a human anatomy database. These samples also include the reconstructed 3D information of the patient's missing region. Naturally, the training samples may undergo certain preprocessing. The corresponding labeled data for these training samples is the reconstructed surface image of the patient's missing region. Similar to 3D reconstruction of missing regions, for the reconstructed surface images of missing areas, it is understood that the construction of training sample-label data can originate from data of patients with actual missing regions or from data of intact human subjects. At minimum, by combining actual and artificial missing conditions, multiple training sample-label data combinations can be constructed for different missing regions under this scenario. This forms the training set used to train the first model reconstruction neural network. The specific training process can be understood by combining missing region reconstruction and conventional neural network training procedures, and will not be detailed here.

In a simplified approach, the surface image obtained for reconstructing the missing region may utilize only surface images near the cross-sectional location of the missing area. By extending this image, a reconstructed surface image of the missing region can be derived, which is also reasonable.

The above provides an example of using the first model reconstruction neural network, where three-dimensional reconstruction of the missing region and surface image reconstruction of the missing region are performed sequentially. In an optional implementation, both steps can be completed in a single process. Specifically, the first model reconstruction neural network is trained using samples constructed from the 3D information of the patient's intact regions and surface images of the remaining intact areas within the human anatomy database. The model is trained using the 3D information and surface images of the missing region to generate label data. Consequently, after training is complete, inputting the 3D information of the patient's remaining areas (excluding the missing region, acquired using depth information acquisition devices) along with the collected surface images of these areas, the first model reconstruction neural network outputs a 3D structural model of the reconstructed missing region, complete with the reconstructed surface image of that region. The embodiments of the present invention may implement the 3D reconstruction of the missing region and the surface image reconstruction of the missing region using either of the aforementioned methods, without limitation. However, it should be emphasized that under different circumstances, the combination of different input information A and output information B utilizes a neural network training process analogous to existing neural network training processes. This embodies the functional principle of neural networks. The focus of the present invention's embodiments lies in constructing a large volume of training samples and labeled data from human anatomical databases based on the objectives represented by input information A and output information B to complete the model training process. However, this process may also be understood by referring to existing neural network training principles and combining them with the scenario objectives of the present invention's embodiments, and no further detailed explanation is provided herein.
Step a2: Perform structural segmentation of internal tissues within the 3D structural model using a pre-built second structural hierarchical neural network. Simultaneously, modify the model using model attribute information extracted from the acquired patient data and a pre-built third model modification neural network. The processed output yields a 3D model.

In existing technology, on one hand, artificially manufactured prosthetic bodies are often limited to approximating the appearance of the natural body at rest. Consequently, completed prostheses are produced through surface painting without internal structural components, typically adopting a monolithic structure lacking differentiation of internal tissues. This results in poor realism for the prosthetic body. After installation in the patient's defect site, the prosthesis and defect surface merely contact at the interface. Internal tissues within the defect, such as bone, cannot be appropriately extended within the prosthesis. Consequently, the prosthesis exhibits poor realism, flexibility, and usability. Furthermore, existing prosthetic body designs and manufacturing processes prioritize industrial scalability. Consequently, identical prosthetic bodies are often used for all patients, with only a limited number of standardized models offered as commercial products for patients to select the closest match. Achieving customization requires manual fabrication by highly specialized personnel, failing to meet current demands.

To address the first issue, the present invention performs structural segmentation of internal tissues within the 3D model of the reconstructed missing area. This enables the differentiation of various tissues within the 3D model—such as bone, muscle, tendon, fat layers, skin layers, etc., within the 3D structural model of the missing area reconstruction. This enables subsequent positioning and material selection for different internal tissues before corresponding printing construction. As a result, the internal tissues within the prosthetic body are differentiated. This not only makes the printed prosthetic body more closely resemble the patient's actual internal structure but also achieves precise alignment matching with the tissue positions corresponding to the missing area's fracture surface. Consequently, the prosthesis can move in a manner closer to that of a real human body.

Specifically, the present invention pre-constructs a second hierarchical neural network for internal tissue segmentation. This second hierarchical neural network, similar to the previously described first model reconstruction neural network, utilizes data from human anatomical databases for training. The selected training samples contain three-dimensional structural models (i.e., 3D information) of reconstructed missing areas, revealing external morphology and internal structures but without distinguishing between tissues such as bones and muscles. These training samples also include at least 3D information of the cross-sectional location of the missing area, or further encompass 3D information of the connecting regions to the missing area.

For example, if a patient has an amputation below the left elbow, the training sample includes a reconstructed 3D structural model of the missing portion below the left elbow, along with 3D information of the cross-section at the left elbow and even the upper arm portion above the cross-section. The corresponding label data for training samples is segmentation information of tissues within the samples—specifically, the three-dimensional data obtained after segmenting the internal tissue structures of the reconstructed 3D models for missing regions. This data represents the three-dimensional shapes of internal structures. Using the example above, it involves manually labeling or otherwise segmenting the reconstructed 3D model of the left elbow-down missing limb to derive the three-dimensional information of internal tissues.

The segmented three-dimensional information for each tissue is obtained by referencing the cross-section and further referencing the three-dimensional information of the remaining upper arm segment connected to the cross-section. This ensures that the shape and position of each tissue segmented within the reconstructed three-dimensional structural model of the missing left limb below the elbow match the shape and position of the corresponding tissue at the cross-section, as well as the shape and position of the corresponding tissue in the remaining upper arm segment connected to the cross-section. For instance, regarding bones, the segmented three-dimensional information encompasses details such as bone thickness, shape, and orientation. Any given bone is aligned with its corresponding bone at the cross-section and with the corresponding bone in the upper arm segment above the cross-section. This ensures that the orientation, shape, and size of the same bone from top to bottom conform to human anatomy, preventing abnormal abrupt changes or misalignment.

Similarly, during the training of the second-structure hierarchical neural network, the construction of training sample-label data can originate from patient data with actual missing regions or from data of intact human subjects. At a minimum, through both actual and artificial missing regions, multiple training sample-label data combinations should be constructed for different missing regions in this scenario. This forms the corresponding training set used to train the second-structure hierarchical neural network at this stage. This can be understood by referencing the descriptions of the first model reconstruction neural network and the principles and training processes of existing neural networks; detailed elaboration is omitted here.

It should be noted that the second-structure hierarchical neural network in this invention's embodiments can be applied to cases where the healthy reference side exhibits asymmetry at the missing site—such as nasal amputation or bilateral arm loss—as well as cases where the healthy reference side exhibits symmetry at the missing site—such as one intact arm and one missing arm. In both scenarios, the training mechanism of the second-structure hierarchical neural network remains identical. The model may be identical across cases or optimized with tailored parameters for different situations—both approaches are valid.

Regarding the second aspect of the problem mentioned above, the shape of the prosthesis should be adjusted according to the patient's specific circumstances. To precisely restore individual variations, the present invention's embodiment again employs artificial intelligence algorithms. It utilizes model attribute-related information from collected patient data and a pre-constructed third model modification neural network to perform model modification. That is, after processing the three-dimensional structural model for the missing area reconstruction through internal tissue segmentation and model modification, the present invention's embodiment ultimately obtains a 3D model.

Similarly, the third model modification neural network in this invention's embodiment can be applied to cases where the healthy reference side of the missing area is asymmetrical and cases where it is symmetrical.

This invention's embodiment does not restrict the sequence of processing between internal tissue segmentation and model modification. For example:

First, perform internal tissue segmentation on the reconstructed 3D structural model of the missing region using a pre-built second structural hierarchical neural network. Then, perform model modification using model attribute information from the acquired patient data and a pre-built third model modification neural network. The final 3D model is obtained after processing. Alternatively, (2) the reconstructed 3D structural model of the missing region can first undergo model modification using the model attribute information from the collected patient data and the pre-built third model modification neural network, followed by internal tissue segmentation using the pre-built second structural hierarchical neural network, ultimately yielding the 3D model; Alternatively, (3) both approaches may be performed in parallel. The processed models from both approaches are then fused or otherwise combined to ultimately generate the 3D model. These three different timing points for the third model modification neural network's involvement are all reasonable. The embodiments of the present invention may adopt any one of these approaches or select the optimal one from among the three.

The model attribute-related information includes:
patient age, gender, height, weight, and skin tone;
Model modification involves adjusting prosthesis size, shape, surface color, and surface texture.

The purpose of model modification is to enhance the alignment between the 3D model and the patient's individual characteristics.

Specifically, when modifying neural networks using the third model, input information A contains the three-dimensional structural model to be processed and related model attribute information. Output information B comprises modification factors such as prosthesis dimensions, shape, surface color, and surface texture, as well as factors like bone thickness, muscle volume, fat layer thickness, skin color, and texture. By applying the obtained modification factors to further refine the three-dimensional structural model, the aforementioned modification objectives can be achieved. The embodiments of the present invention can utilize extensive data to predefine modification standards, such as criteria for various human modification factors under attributes like different ages and skin tones. Modification factors are used to modify both external and internal structures. For instance, model attribute-related information may include details like the patient being a 70-year-old Asian female. Modification factors can be described numerically or verbally, such as: Bone width ML_width_of_bone=0.95, Skin color

Mcolor_RGB=0.9,1.2,1.1, etc. These modification factors can then be applied during prosthesis manufacturing processes, such as 3D printing and spray painting.

The process of utilizing the third-model-modified neural network varies depending on the timing of its involvement. Consequently, the three-dimensional structural model to be processed differs, and the training samples and labeled data for its training process also change accordingly.

For instance, in the participation scenario indicated by (1) above, the 3D structural model processed by the third-model-modified neural network is obtained as follows: the reconstructed 3D structural model of the missing region undergoes internal tissue segmentation using a pre-built second-layer structural neural network. The third-model-modified neural network then outputs modification factors. Adjusting the processed 3D structural model with these modification factors yields the final 3D model.

In scenario (1), the training sample data includes: A large number of 3D structural models reconstructed from missing regions, obtained from human anatomical databases, which undergo internal tissue segmentation to yield the final 3D structural models; Corresponding model attribute information from patient records. Label data includes corresponding modification factors. Training sample-label data construction may originate from data of patients with actual missing regions or from data of intact human subjects. At minimum, through both real and artificial missing region scenarios, multiple training sample-label data combinations should be constructed for different missing regions under this scenario. This forms the training set used to train the third model modification neural network. The choice of network architecture for the third model modification neural network is unrestricted. and can be understood by referencing the relevant descriptions of the first model reconstruction neural network and other networks, as well as the principles and training process of existing neural networks. Further details are omitted here.

Regarding the participation timing indicated by (2) above, the three-dimensional structural model to be processed by the third model-modified neural network is the reconstructed three-dimensional structural model of the missing region. The third model-modified neural network outputs a modification factor. After adjusting the three-dimensional structural model to be processed using the modification factor, a modified three-dimensional structural model is obtained. This modified model undergoes internal tissue structural segmentation using the pre-constructed second structural hierarchical neural network, thereby yielding the described 3D model. In this scenario, the sample data and label data for the training process can be understood in relation to the model usage process. This can be understood in conjunction with the above scenario (1), the relevant descriptions of networks such as the first model reconstruction neural network, the principles of existing neural networks, and the training process. Detailed explanation is omitted here.

For the participation timing indicated by (3) above, the three-dimensional structural model to be processed by the third model modification neural network is the three-dimensional structural model reconstructed for the missing region; The third model modifies the output modification factors of the neural network. After adjusting the 3D structural model to be processed using these modification factors, a modified 3D structural model is obtained. This modified 3D structural model is then fused with the 3D structural model obtained from the segmentation of internal tissue structures using the pre-built second hierarchical structural neural network. Through model fusion or similar processing methods, the 3D model is obtained. The specific methods for model fusion can be implemented based on existing technologies. In such scenarios, the sample data and label data used in the training process can be understood in the context of model application. This can be comprehended by combining the aforementioned Scenario (1), the relevant descriptions of the first model reconstruction neural network and other networks, as well as the principles and training processes of existing neural networks. Detailed explanations are omitted here.
Step a3: Perform a two-dimensional image unfolding on the reconstructed surface image of the missing part based on the 3D model to obtain a 2D unfolded image.

As mentioned earlier, the reconstructed surface image of the missing area represents the surface of its three-dimensional structure. Therefore, it constitutes a three-dimensional surface image. Existing relevant technologies, such as common software like Adobe Illustrator or 3D printer software, can be used to directly unfold this three-dimensional surface image into a two-dimensional image. This process is analogous to unfolding a scroll, yielding a two-dimensional image. After undergoing certain processing steps, the 2D unfolded image is obtained. The 2D unfolded image serves as the printable data for 2D printing devices.

Furthermore, to generate more realistic 2D unfolded images for printing, this invention's embodiments design a reverse filtering algorithm for two scenarios: when the missing region lacks a symmetrical healthy reference side, and when it possesses a symmetrical healthy reference side. For clarity, this section is detailed below.

(2) For cases where the missing region has a symmetrical healthy reference side, the main computer acquires patient information and utilizes a pre-built artificial intelligence model to obtain a 3D model containing segmentation information of each tissue within the patient's missing region. Based on the acquired patient information and the 3D model, it generates a 2D unfolded image for the surface layer of the 3D model, including:
Step b1: The main computer uses a depth information acquisition device to collect depth information of the healthy reference side and the remaining portion of the missing area. It also uses an image acquisition device to collect multiple surface images of the healthy reference side at different angles.

Specifically, the main computer controls the depth information acquisition device to collect depth information—i.e., three-dimensional information—of the patient's healthy reference side and the remaining portion of the missing area. The depth information acquisition device is as described earlier.

Since this scenario involves reconstructing the three-dimensional structure of the missing side using the symmetrical healthy reference side, it is necessary to collect depth information from the healthy reference side as a reference basis. Similar to cases where no symmetrical healthy reference side exists for the missing area, it is also necessary to collect at least the three-dimensional information of the cross-sectional position of the missing area, or further, the three-dimensional information (i.e., depth information) of the area connected to the missing area. This enables the acquisition of the boundary region structure between the remaining and missing parts of the patient's body, which is used to design the wear portion of the prosthesis body.

In step b1, the main computer controls the image acquisition device to capture multiple surface images of the healthy reference side at different angles. The image acquisition device is as described earlier.
Step b2: Utilize the acquired depth information to perform three-dimensional reconstruction of the missing area, yielding a reconstructed three-dimensional structural model.

For example, if the patient's left arm is missing below the elbow joint while the right arm remains intact, this scenario employs the right arm to reconstruct the three-dimensional structural model of the missing portion of the left arm below the elbow joint. The complete 3D information of the right arm can be captured, as this data represents the intact three-dimensional shape and structure of the right arm. Utilizing at least this 3D information for the left arm's reconstruction yields a reconstructed 3D structural model of the left arm.

During this reconstruction process, leveraging the symmetrical reference side, existing algorithms and models can be applied in conjunction with human anatomy principles to construct a symmetrical three-dimensional structural model of the reconstructed left arm. This reconstructed model exhibits symmetrical external structure relative to the right arm.
Step b3: Perform internal tissue segmentation on the 3D structural model using a pre-built second structural hierarchical neural network. Additionally, modify the model using attribute-related information from the acquired patient data and a pre-built third model modification neural network. The processed output yields a 3D model.

In step b3, the reconstructed 3D structural model of the missing side and the 3D information of at least the cross-section of the missing area undergo further processing to yield input information A for the second structural hierarchical neural network at that location. The output information B is the 3D structural model obtained after segmenting the internal tissue structure of the input 3D structural model.

The processing in step b3 is analogous to step a2; refer to step a2 for details.

It should be noted that for cases where the healthy reference side is asymmetric relative to the missing region and for cases where the healthy reference side is symmetric relative to the missing region, the second structural hierarchical neural network can share the same network architecture but employ distinct network parameters. These parameters are determined and saved through separate training processes for each scenario. When utilizing the second structural hierarchical neural network for these two distinct cases, the corresponding network parameters can be invoked to generate the appropriate output information B for the input information A specific to that scenario. The process of invoking adapted network parameters can be implemented via computer programs.

Similar to the second-layer hierarchical neural network, for cases where the healthy reference side lacks symmetry relative to the missing region and for cases where the healthy reference side exhibits symmetry relative to the missing region, the third-model modification neural network can also share the same network architecture but employ distinct network parameters. These parameters are invoked correspondingly during usage.
Step b4: Perform two-dimensional image unfolding on the acquired multiple surface images based on the 3D model to obtain 2D unfolded images.

For cases where the defect has a symmetrical healthy reference side, the collected surface images can be processed using existing methods to generate a surface image representing the defect's 3D structure based on the 3D model and symmetry principles. This 3D surface image is then directly unfolded into a 2D image using the same method as in step a3. After processing, the resulting 2D unfolded image is obtained. The 2D unfolded image is the image used for printing by 2D printing equipment. Such equipment may include 2D printers, etc.

One feature of this application is the provision of an automated surface printing technique. In this technique, the surface image of the missing area is printed onto the manufactured surface membrane of the missing area using a 2D printing device to form a skin covering. This surface membrane of the missing area serves as a simulated skin membrane. Due to differences in color filters between the camera and other devices used to capture the surface image and the 2D printing device, slight variations in printed color, texture, and other aspects compared to the actual surface are unavoidable. To output more realistic 2D digital images for printing, the present invention's embodiments jointly design a reverse filtering algorithm for cases where the affected area lacks a symmetrical healthy reference side and for cases where it possesses such a reference side. This algorithm is applied to the digital image prior to printing to resolve color discrepancies between the printed image and the actual object. The following briefly outlines the primary implementation principle of this algorithm.

Capture an image of the test sample using a digital camera to obtain the test image, expressed by the formula:

$$I test = i test * f camera ;$$

The test sample referred to herein generally denotes a physical object, which may be a human subject. It should be understood that the test image is a digital image.

In the embodiments of the present invention, i(x, y) denotes a visual image, I(x, y) denotes a digital image, where (x, y) refers to the coordinates of a pixel; f(x, y) denotes the filter function; In the above formula, I test denotes the test image; i test denotes the visual image of the test sample; f camera denotes the filter function of the digital camera;

The test image I test is printed using a 2D printing device to obtain the corresponding printed image, expressed by the formula:

$$i test , print = I test * f printer = i test * f camera * f printer ;$$

Here, i test, print denotes the printed image obtained by printing the test image I test using a 2D printing device; f printer denotes the filtering function of the 2D printing device.

3. By simultaneously photographing the test image and its printed image using the same digital camera, the following relationship can be obtained:

$$I test = i test * f camera ;$$

$$I test , print = i test , print * f camera = i test * f printer * f {camera}^{2} ;$$

Among these, the I test, print is a digital image obtained by photographing the printed test image with a digital camera;

4. Calculate the correction factor using the digital images obtained by photographing the test image and its printed image with a digital camera, expressed as:

$$α ( x , y ) = {I}_{test} {I}_{test , print} = {( {f}_{printer} * {f}_{camera} )}^{- 1} ;$$

Here, [symbol] denotes a two-dimensional deconvolution operation, and * denotes a two-dimensional convolution operation.

Understandably, both the test image and its printed image, when captured by a digital camera, yield digital images. The correction coefficient is calculated as the ratio between the two, where α(x, y) denotes the corresponding correction coefficient at pixel location (x, y).

5. Using the same digital camera to capture images of the target human body part, represented as:

$${I}_{body} = {i}_{body} * {f}_{camera} ;$$

In this context, I body denotes the target human body part image, which is also a digital image; i body denotes the corresponding visual image.

6. After applying correction coefficients to the target body part image I body, it is printed using a 2D printing device to obtain the corresponding printed image, represented as:

$${i}_{print , out} = {I}_{body} * α ( x , y ) * {f}_{printer} ;$$

Among these, i_{print,out} represents the final printed image corresponding to the target human body part image. i_{print,out} equals i body through compensation by correction coefficients.

It is evident that deviations between printed output and the true original image are unavoidable during digital image acquisition and printing. However, the present invention's embodiments address this by measuring correction coefficients within the digital image. Finally, by multiplying the correction coefficients stored in the computer prior to printing, the printed output becomes a true image with the difference pre-compensated.

Building upon the aforementioned core implementation principle, the reverse filtering algorithm is elaborated as follows. For the present invention's embodiment, the process of obtaining the 2D unfolded image employs a predefined reverse filtering algorithm. By comparing the digital image of the 3D model's surface layer with its printed image obtained via 2D printing equipment, the algorithm compensates for the perceptual visual differences between the digital image and its printed counterpart, yielding the 2D unfolded image ready for printing.

wherein the digital image comprises: when the missing part lacks a symmetrical healthy reference side, the image obtained by 2D unfolding the surface image reconstructed from the 3D model for the missing part; or when the missing part has a symmetrical healthy reference side, the image obtained by 2D unfolding multiple collected surface images based on the 3D model.

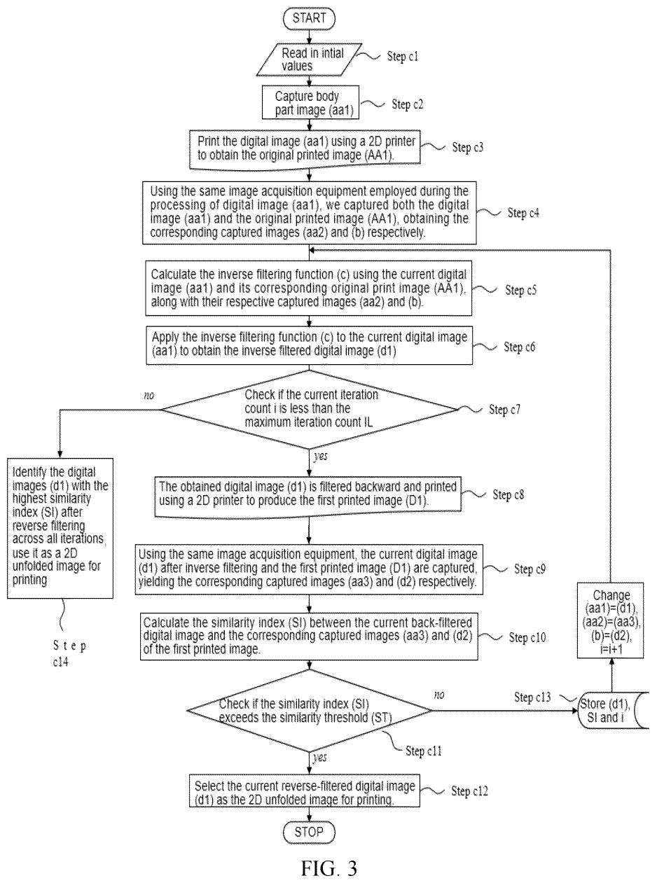

Specifically, refer to FIG. 3. In the embodiment of the present invention, the process of the preset inverse filtering algorithm includes:
Step c1: Reading initial parameters;
wherein the initial parameters include image matrix size, similarity threshold, and maximum iteration count;

Image matrix size refers to the image matrix dimensions of the digital image and printed image, denoted as IM; similarity threshold is denoted as ST; iteration count is denoted as a positive integer i, where i=1 for the first iteration; maximum iteration count is denoted as IL, with IL≥2.
Step c2: Acquire the digital image;

As described earlier, for cases where the missing region lacks a symmetrical healthy reference side, the digital image is the two-dimensional unfolded image of the surface image reconstructed from the 3D model for the missing region;

For cases where the missing region has a symmetrical healthy reference side, the digital image is the two-dimensional unfolded image of multiple acquired surface images reconstructed from the 3D model.

For clarity, the digital image is denoted as (aa1) in FIG. 3.
Step c3: Print the digital image using a 2D printing device to obtain the original printed image;

For clarity, the original printed image is denoted as (AA1) in FIG. 3.
Step c4: Using the same image acquisition device employed in obtaining the digital image, capture the digital image and the original printed image to obtain corresponding captured images.

The image acquisition device may be a digital camera; the front and rear configurations may be identical.

For clarity, FIG. 3 illustrates the acquisition of the digital image (aa1) and the original printed image (AA1), yielding the corresponding acquired images (aa2) and (b).
Step c5: Calculate the inverse filtering function using the acquired images corresponding to the current digital image and the original printed image;

Specifically, compute the inverse filtering function using (aa2) and (b), expressed by the formula:

$$( aa 2 ) ( b ) = ( c ) ;$$

Here, [symbol] denotes a two-dimensional deconvolution operation, and * denotes a two-dimensional convolution operation. (c) denotes the inverse filtering function.

Based on the preceding explanation of the core implementation principles of this inverse filtering algorithm, the inverse filtering function computed in step c5 serves the same purpose as the correction coefficient described earlier.
Step c6: Apply the inverse filtering function to the current digital image to obtain the inverse-filtered digital image.

Specifically, applying the inverse filtering function (c) to the current digital image (aa1) yields the inverse-filtered digital image (d1), expressed as:

$$( aa 1 ) * ( c ) = ( d 1 ) ;$$

Step c7: Determine whether the current iteration count is less than the maximum iteration count;

Specifically, determine whether the current iteration count i is less than the maximum iteration count IL. If so, proceed to step c8; if not, proceed to step c14.
Step c8: Print the currently obtained reverse-filtered digital image using a 2D printing device to obtain the first printed image. Referring to FIG. 3, the currently obtained reverse-filtered digital image (d1) is printed using a 2D printing device to obtain the first printed image, denoted as (D1).
Step c9: Using the same image acquisition device, capture images of the current reverse-filtered digital image and the first printed image to obtain corresponding captured images.

In FIG. 3, capturing images of the current reverse-filtered digital image and the first printed image yields corresponding captured images (aa3) and (d2).
Step c10: Calculate the similarity index for the captured images corresponding to the current reverse-filtered digital image and the first printed image.

Specifically, calculate the similarity index SI for (aa3) and (d2). The similarity index SI may be implemented using any existing method for calculating image similarity, such as PSNR (Peak Signal-to-Noise Ratio), SSIM (Structural Similarity Index), MSE (Mean Squared Error), cosine similarity, histogram, mutual information, hash similarity, etc., without limitation herein. For example, the similarity index SI could be a two-dimensional correlation coefficient.
Step c11: Determine whether the similarity index exceeds the similarity threshold;

Specifically, determine whether the similarity index SI is greater than the similarity threshold ST. If yes, proceed to step c12; if no, proceed to step c13.
Step c12: Determine the currently obtained reverse-filtered digital image as the 2D unfolded image to be printed;

Specifically, determining the currently obtained reverse-filtered digital image (d1) as the 2D unfolded image to be printed means that the first printed image (D1) obtained from printing it meets the requirements, and (D1) can be used as the final output.
Step c13: Store the current inverse-filtered digital image and similarity index. Replace the current digital image with the current inverse-filtered digital image, replace the current digital image's corresponding captured image with the inverse-filtered image's corresponding captured image, and replace the current original printed image's corresponding captured image with the current first printed image's corresponding captured image. Then return to step c5 for a new iteration.

Specifically, store the currently obtained reverse-filtered digital image (d1), similarity index SI, and current iteration count i;

Then make the following changes: (aa1)=(d1); (aa2)=(aa3); (b)=(d2); i=i+1; Afterward, return to step c5 for a new iteration.
Step c14: Locate the digitally filtered image with the highest similarity index across all iterations and designate it as the 2D unfolded image for printing.

Following steps c12 and c14, the 2D unfolded image can be printed using a 2D printing device. It is understood that the resulting printed image corresponds to (D1).

The reverse filtering algorithm designed in this embodiment eliminates color differences between the printed image and the physical object, enhancing the authenticity of the printed image.
S2, the main computer controls the 3D printing device to print the 3D model, obtaining the prosthesis body;

The 3D printing device may be a 3D printer, etc.

As previously described, the 3D model contains three-dimensional information of the missing region and has undergone segmentation of internal tissues such as bones, muscles, tendons, fat layers, and skin layers. Therefore, the three-dimensional information of each tissue—including shape, structure, size, and position—is known, enabling 3D printing via the 3D printing device. Each part can be printed separately, with the main computer controlling an assembly device to assemble the prosthetic body. That is, the prosthetic body comprises the printed results of the layered parts corresponding to the missing region.

In one optional implementation, S2 may include:
The main computer controls the 3D printing equipment to print using materials matched to each tissue in the 3D model, thereby producing the prosthetic body; wherein the tissue-matched materials comprise a variety of optional materials.

Considering the use of the prosthesis body, materials for each tissue type should be selected with care. The present invention may pre-conFIG. multiple material options for each tissue type by referencing general human (mechanical) parameter values provided in user manuals and literature, such as physical parameter values (hardness and viscoelasticity, etc.) for a wide range of human tissues. These materials must also be capable of long-term repeated use and possess biocompatibility. When printing the 3D model, one material is selected from the multiple options for each tissue type—for example, through random selection—to print that tissue. Upon completing printing for all tissues, the printed prosthesis body is obtained.

Furthermore, patient-specific information can be integrated to tailor material selection for each tissue. Specifically, for each tissue type, the matched material is determined from its set of options based on collected patient data, ensuring it closely mimics the actual patient's tissue in terms of hardness, viscoelasticity, and other properties. For this scenario, physical parameter values such as hardness and viscoelasticity can be collected for each patient tissue at any stage prior to 3D model printing. Subsequently, for each tissue type, a suitable material is selected from its multiple options using the collected patient parameters for printing. For instance, plastic materials can be used to print components like bones, muscles, and fat layers, but different plastics can be selected for each tissue based on the patient's physical parameter values. Furthermore, the size of subcutaneous components such as bones, muscles, tendons, and fat layers can be adjusted according to skin thickness.
S3, wherein the main computer controls the film-making device to manufacture a surface film for the missing area;

The surface film for the missing area is a simulated skin membrane, i.e., a membrane mimicking real human skin. Its mechanical properties (such as hardness and viscoelasticity) must match the remaining part of the patient's limb and possess biocompatibility. Silicone rubber is one of the candidate materials.

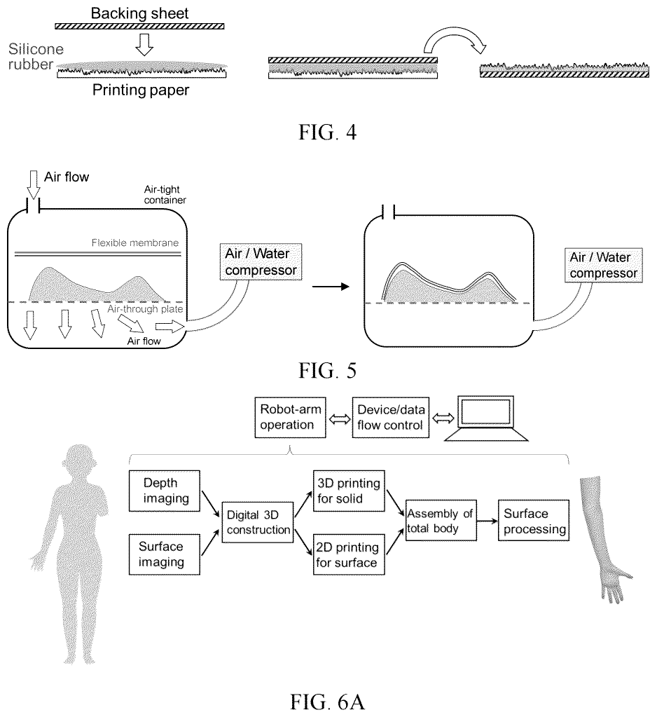

In an optional implementation, refer to the schematic diagram of the missing area surface membrane manufacturing process shown in FIG. 4 for understanding. S3 may include the following steps:
S31, the main computer controls the film-making equipment to obtain a sol-state silicone rubber solution, which is placed on printing paper with a preset thickness and surface roughness to form a silicone rubber solution layer of the set thickness;
wherein the film-making device may be implemented based on existing process equipment, featuring a platform for placing printing paper. The printing paper possesses a predetermined thickness and surface roughness, with the predetermined thickness being a thickness value determined based on the thickness of a large number of human skin samples, and the predetermined surface roughness being a roughness value determined based on the roughness of a large number of human skin samples, thereby adapting to most human subjects.

In an optional embodiment, to customize for patients requiring prostheses, the preset surface roughness is determined based on the skin surface roughness collected from the patient's information, and the preset thickness is determined based on the skin thickness collected from the patient's information. In this case, the surface roughness and thickness of the patient's skin must be collected prior to step S3, serving as the preset surface roughness and preset thickness, respectively.

After the film-forming device obtains the sol-state silicone rubber solution, it is placed on the printing paper to uniformly cover the surface with a set thickness, forming a silicone rubber solution layer of the specified thickness. Refer to the lower portion of the leftmost diagram in FIG. 4: the burrs on the printing paper represent the rough surface, while the blue area indicates the formed silicone rubber solution layer.
S32, the main computer controls the film-forming equipment to cover the substrate sheet on the upper surface of the silicone rubber solution layer. After the silicone rubber solution layer gels, it is peeled off to obtain the surface film with missing areas. The original contact surface between the surface film with missing areas and the printing paper serves as the printing surface; wherein the substrate sheet exhibits higher adhesion strength to the silicone rubber than the printing paper surface, and the printing paper is compatible with the 2D printing device.

The backing paper pad in FIG. 4 serves as the substrate sheet. The substrate sheet's surface exhibits higher adhesion strength to silicone rubber than the printing paper's surface, facilitating subsequent peeling and removal of the printing paper.

After covering the upper surface of the silicone rubber solution layer with the substrate sheet, the structure shown in the middle portion of FIG. 4 is formed. Once the silicone rubber solution layer gels, it is peeled off from the printing paper surface. After flipping, the structure shown in the right portion of FIG. 4 is obtained, yielding the surface film for the missing area. The original contact surface between the printing paper and the substrate sheet serves as the printing surface. Through the above process, a silicone rubber film with the same surface roughness as the printing paper can be produced. Subsequently, a standard 2D printing device can be used to print patterns and apply color to the surface film of the missing area, thereby forming a skin.
S4, the main computer controls the 2D printing device to print the 2D unfolded image onto the surface film of the missing area to form a skin;

In an optional embodiment, prior to executing S4, the present method further comprises:

The main computer controls the printing system to treat the surface of the surface film of the missing area with a surface treatment solution and uniformly print pigment ink after treatment.

The printing system may include 2D printing equipment and 3D printing equipment, and may also include other equipment. It may be implemented based on combinations of existing equipment capable of achieving the functions described herein, and will not be detailed herein.

Treating the surface of the top layer film at the missing area with the surface treatment liquid involves physically/chemically treating the surface to enhance affinity with the ink. The surface treatment liquid may utilize existing liquids for surface treatment and is not limited herein.

Following treatment, the printing system uniformly prints pigment ink onto the surface of the surface film of the missing area treated with the surface treatment liquid. This facilitates subsequent step S4, where the 2D unfolded image is printed onto the pigment-inked surface film of the missing area, forming a patterned skin.

When performing step S4, the 2D printing device may utilize a standard inkjet printer. Thus, the process described in the present invention enables printing on silicone rubber film using conventional inkjet printers, significantly reducing production costs.

Furthermore, due to silicone rubber's low ink permeability, pigment-based inks are more suitable than dye-based inks for general printing.

Following S4 printing, a protective ink layer must be applied to the printed surface while ensuring it matches the texture of real human skin.

This constitutes the final coloring process, aimed at achieving similarity to the actual body.

Silicone rubber is widely used in prosthetic materials due to its physical, chemical, and biological inertness, along with its stability in everyday environments. However, its inert nature makes direct printing on silicone rubber surfaces challenging for standard inkjet or laser printers. The present invention proposes a simple and practical technique to achieve high-quality printing on silicone rubber films. By treating the silicone rubber film surface with a surface treatment solution and uniformly applying pigment ink (base coat), a 2D unfolded image of the patient's missing body part can be printed on top. This simplifies the process and reduces manufacturing costs.
S5: The main computer controls the assembly equipment to bond the skin to the surface of the prosthesis body. After surface treatment, the prepared prosthesis is obtained.

As previously described, with all components of the prosthesis body already assembled, the skin is directly bonded to the surface of the prosthesis body to obtain the prepared prosthesis, i.e., the finished prosthesis product.

In an optional embodiment, the main computer-controlled assembly device bonding the skin to the surface of the prosthesis body may include:

The main computer controlling the printing system applies adhesive and lubricant to the surface of the prosthesis body. The covering material is then placed over the coated surface of the prosthesis body. An even pressure application device is controlled to apply pressure, causing the covering material and the prosthesis body to adhere tightly and form an integrated structure.

In this embodiment of the invention, assembly equipment is used to bond the covering material to the prosthesis body. The assembly equipment may include robotic arms, etc.

To simulate the adhesive state of real human skin, the density and adhesion strength of the adhesive match the collected patient data. Specifically, the adhesive possesses the same viscoelastic properties as the surface membrane of the missing area and the patient's body. The adhesive may be a resin-based adhesive, whose density and adhesion strength can be pre-adjusted to align with the patient's body data.

The lubricant employs a biocompatible lubricant, such as an inert oil, to achieve the natural lubrication effect of the human body.

To achieve a highly realistic reproduction of the human skin surface, the present invention provides a uniform pressure application device designed to apply even pressure across all sections of the prosthetic body covered by the skin-like membrane. This ensures a secure bond between the membrane and the prosthetic body, preventing internal voids and surface wrinkles. The operating principle of this uniform pressure application device is illustrated in FIG. 5.

In FIG. 5: Air flow denotes air flow; Air-tight container denotes an airtight container; Flexible membrane denotes the flexible membrane, i.e., the skin in this embodiment; Air-through plate denotes an air-permeable plate; Air/Water compressor denotes an air/water compressor;

The operation of this uniform pressure application apparatus may include:
1. Placing the solid model on the air-permeable plate and positioning the flexible membrane above the model. They are placed within a container featuring airflow (or water flow) inlet and outlet ports. A layer of adhesive is applied to the surface of the solid model or beneath the flexible membrane.
2. By altering the internal air pressure through high-pressure air or flowing water injected from the inlet, the flexible membrane is pressed tightly against the surface of the solid model.
3. Since pressure is uniformly applied to the membrane within this structure, the membrane can be adhered uniformly and tightly to the surface of the solid model.

Here, the solid model refers to the prosthesis body, and the flexible membrane refers to the skin.

After bonding the skin to the prosthesis body surface, surface treatment is required to produce the finished prosthesis.

The surface treatment here includes:
The main computer-controlled spray system applies a protective layer resembling human skin to the surface of the bonded prosthesis body.

The spray system may be implemented using existing equipment or combinations thereof, provided it achieves the described function without limitation.

The protective layer resembling human skin can be pre-determined based on relevant patient skin surface data. After application, the prosthesis's outermost layer exhibits identical texture, diffuse reflectance, and water/oil affinity to genuine patient skin, along with sufficient durability for daily use. The protective layer material is unrestricted and may include silicone-based resins, among others.

It is understood that the present invention's embodiment employs a main computer to control the operation of other components, monitor and control data flow, thereby automating the following process: Construction of a 3D model containing segmentation information of tissues within the patient's missing area→Generation of a 2D unfolded image of the 3D model's surface→3D printing of the prosthesis body→Fabrication of the surface membrane for the missing area→Printing the 2D unfolded image onto the surface membrane to form a skin layer→Adhering the skin layer to the surface of the prosthesis body, followed by surface treatment to obtain the completed prosthesis.

Throughout this process, a robotic arm controlled by the main computer can be used to move and manipulate necessary equipment, tools, and various components of the prosthesis. Other components controlled by the main computer include, but are not limited to, the robotic arm, as well as the aforementioned printing systems (containing 3D and 2D printing equipment), membrane fabrication equipment, assembly equipment, depth information acquisition equipment, image acquisition equipment, uniform pressure application instruments, and spray painting systems. The main computer also controls data stream transmission for RGBD images, 3D internal structure point datasets, AI network requirements (e.g., training data), 3D printing files (OBJ, STL engineering files), and robotic arm control for assembly and surface treatment.

The fully computer-intelligent high-similarity prosthesis production method provided by the present invention's embodiment comprehensively computerizes and intelligently integrates all aforementioned processes. This enables the provision of high-precision, high-similarity prostheses within shorter timeframes. By digitizing the production workflow through computer operation, reliance on manual experience or skills is eliminated, significantly reducing labor time and costs.

Furthermore, this invention incorporates artificial intelligence technology combined with patient information for prosthesis design and modeling. Utilizing AI not only generates high-precision, high-fidelity 3D models containing segmentation data of tissues within the patient's missing area for 3D printing output—enabling segmentation of internal limb structures (such as bones, muscles, tendons, fat layers, skin layers, etc.) and reconstruction of missing structural components—It also enables the generation of highly realistic 2D unfolded images of the 3D model's surface for 2D printing output.

Through artificial intelligence algorithms, prosthetic design and production can be achieved not only by utilizing the healthy reference side as data when a symmetrical healthy reference side exists for the missing area, but also by restoring limbs even when both symmetrical body parts (hands, arms, legs, feet, ears, etc.) are missing. Furthermore, prosthetic models can be generated for single-site defects (nose, chin, etc.). Compared to traditional methods, this approach enables more effective customization of prostheses for individual patients, achieving high similarity between the prosthesis and the patient's body, thereby enhancing prosthesis quality. It eliminates limitations in prosthesis production for missing body parts, meets the needs of more patients, and increases the practicality of this method.

Second, corresponding to the aforementioned method embodiments, the present invention further provides a fully computer-intelligent high-similarity prosthetic production system, comprising:
a main computer and other components; wherein the main computer controls the other components to perform the steps of the fully computer-intelligent high-similarity prosthetic production method described in the first aspect; the other components include an image acquisition and storage system, a mold-making device, an assembly device, a printing system, a uniform pressure application instrument, and a spray painting system; wherein the image acquisition and storage system comprises depth information acquisition equipment and image acquisition equipment; and wherein the printing system comprises at least 3D printing equipment and 2D printing equipment.

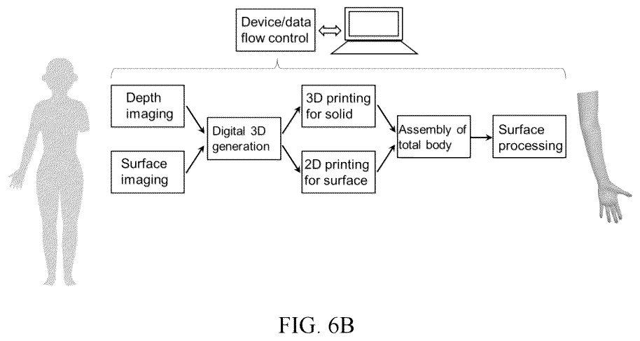

Combined with the foregoing, it can be understood that the present invention can complete prosthesis production for missing body parts in both scenarios: where the missing part has a symmetrical healthy reference side, and where the missing part lacks a symmetrical healthy reference side. To provide a more intuitive understanding of the operational process of this fully computer-intelligent high-similarity prosthetic production system under these two scenarios, a brief explanation is provided below using FIG. 6A and FIG. 6B as examples, in conjunction with the content of the first aspect described earlier. FIG. 6A and FIG. 6B illustrate the entire operational process of the system for producing fully digital prosthetics (such as hands, arms, legs, breasts, ears, etc.). FIG. 6A addresses cases where the missing part has a symmetrical healthy reference side, i.e., when one side of the symmetrical body part remains intact. FIG. 6B addresses cases where the missing part lacks a symmetrical healthy reference side, i.e., when both sides are missing or no symmetrical part exists.

For both FIG. 6A and FIG. 6B, the computer icon at the top represents the main computer. In the workflow below, each step involves additional components that are not shown for simplicity. Throughout the system's operation, the main computer controls these components and manages the corresponding data flow.

For FIG. 6A and FIG. 6B, the steps following digital 3D modeling are identical. Below is a brief description of each step.

Digital 3D Modeling: This step generates two outputs. First, it produces a 3D model containing segmentation information of tissues within the patient's missing region, which is provided to the 3D printing device for subsequent 3D skeletal structure printing. Second, it generates a 2D unfolded image of the 3D model's surface layer, supplied to the 2D printing device for subsequent 2D surface printing. For specific details on this section, please refer to the relevant content in SI above.

Specifically, for FIG. 6A, digital 3D modeling utilizes depth images and surface images. The depth images refer to capturing depth information from the patient's healthy reference side and the remaining portion of the missing area. The surface images refer to capturing multiple surface images of the healthy reference side at different angles. Subsequently, the collected depth information is used to perform three-dimensional reconstruction of the missing area, yielding a reconstructed three-dimensional structural model.

Regarding FIG. 6B, the digital 3D modeling was accomplished using generative AI. Here, generative AI primarily refers to the reconstruction neural network built upon a pre-established first model. By collecting information from the patient's remaining body parts, it performs three-dimensional reconstruction of the missing areas, yielding a reconstructed 3D structural model and surface images of the reconstructed missing regions.

In traditional prosthesis fabrication, designers reference remaining body parts to create symmetrical components such as arms, legs, hands, feet, breasts, and ears. However, perfectly symmetrical appearances often appear unnatural. Designers therefore introduce subtle asymmetrical modifications to enhance naturalness. Such adjustments rely heavily on the artisan's advanced skills and extensive experience. In the embodiments of the present invention, deep learning principles can be employed to achieve these modifications. Within the digital system's data management, the invention utilizes a large-scale human anatomy database to train a neural network system. This network outputs prostheses with natural appearances based on the asymmetry of body parts. Furthermore, the network of this invention possesses a significant advantage. Utilizing generative AI principles, it can generate missing body parts without reference to residual structures. Consequently, it can produce prostheses lacking symmetrical structures, such as noses or chins. Additionally, it can generate prostheses for bilateral amputations, such as those for missing legs.

For both scenarios, the internal tissue structure segmentation of the 3D model for missing part reconstruction is performed using a pre-built second-layer hierarchical neural network. Model modification is then executed using model attribute information from the acquired patient data and a pre-built third-layer model modification neural network, yielding the processed 3D model. Additionally, the corresponding surface images of the missing parts in each scenario undergo 2D image unfolding to produce 2D unfolded images.

For specific details, please refer to the relevant descriptions in the preceding text; no further elaboration is provided here.

3D Skeletal Structure Printing: This section refers to the preceding S2 section, wherein the main computer controls the 3D printing device to print the 3D model, yielding the prosthesis body.

2D Surface Printing: Refer to Sections S3 and S4 above. First, the main computer controls the film-making device to produce a surface film for the missing area. Then, the main computer controls the 2D printing device to print the 2D unfolded image onto the surface film, forming a skin.

Overall Structure Assembly and Surface Treatment: Refer to Section S5 above. The main computer controls the assembly equipment to bond the skin to the surface of the prosthesis body. After surface treatment, the prepared prosthesis is obtained.

For specific processing procedures of each system module, refer to the relevant content in Aspect I. Further details are omitted here.

The fully computer-intelligent high-similarity prosthesis production system provided by the embodiments of the present invention achieves comprehensive computerization and intelligence throughout the prosthesis manufacturing process. It enables the provision of high-precision, high-similarity prostheses within shorter timeframes. By digitizing the production workflow through computer operation, it eliminates reliance on manual experience or skills, significantly reducing labor time and costs.

Furthermore, by integrating artificial intelligence technology with patient data for prosthesis design and modeling, enabling prosthetic design and production using the healthy reference side as data when a symmetrical healthy reference side exists for the missing area. Moreover, it can restore limbs even when both symmetrical body parts (hands, arms, legs, feet, ears, etc.) are missing, and generate prosthetic models for single-site defects (nose, chin, etc.). Compared to traditional methods, this approach enables more effective customization of prostheses for individual patients, achieving high similarity between the prosthesis and the patient's body, thereby enhancing prosthesis quality. It eliminates limitations in prosthesis production for missing body parts, meets the needs of more patients, and improves practicality.

Thirdly, corresponding to the fully computer-intelligent high-similarity prosthetic production method provided in the first aspect, the present invention also provides a computer-readable storage medium. This storage medium contains a computer program that, when executed by a processor, implements the steps of any fully computer-intelligent high-similarity prosthetic production method provided in the first aspect of the present invention.

Specific details may be referenced in the fully computer-intelligent high-similarity prosthesis production method provided in the first aspect and are not repeated herein.

It should be noted that in the description of the present invention, the terms “first” and “second” are used solely for descriptive purposes and should not be construed as indicating or implying relative importance or as implying the number of technical features indicated. Thus, features defined as “first” or ‘second’ may explicitly or implicitly include one or more such features. In the description of the present invention, “multiple” means two or more, unless otherwise explicitly and specifically defined.

In the description of this specification, references to “an embodiment,” “some embodiments,” “an example,” “a specific example,” or “some examples” indicate that the specific features, structures, materials, or characteristics described in connection with that embodiment or example are included in at least one embodiment or example of the present invention. In this specification, the illustrative use of the above terms need not be directed to the same embodiment or example. Moreover, the specific features, structures, materials, or characteristics described may be appropriately combined in any one or more embodiments or examples. Furthermore, those skilled in the art may combine and integrate different embodiments or examples described herein.

Each embodiment in this specification is described in a relevant manner. Identical or similar parts among embodiments may be cross-referenced, with each embodiment focusing on highlighting differences from others. Particularly for system and storage medium embodiments, as they are fundamentally similar to method embodiments, their descriptions are relatively concise; relevant aspects may be referenced in the corresponding sections of the method embodiments.

The foregoing description is intended to illustrate preferred embodiments of the present invention and is not intended to limit the scope of protection of the invention. Any modifications, equivalent substitutions, improvements, or other variations made within the spirit and principles of the invention are included within the scope of protection of the invention.

1. A fully computer-intelligent method for producing highly similar prostheses, characterized by comprising: The main computer acquires patient information and utilizes a pre-built artificial intelligence model to generate a 3D model containing segmentation information of tissues within the patient's missing area. Based on the acquired patient information and said 3D model, it obtains a 2D unfolded image of the 3D model's surface layer; wherein the tissues include bone, muscle, tendon, fat layer, and skin layer. wherein the tissues include bone, muscle, tendon, fat layer, and skin layer;
The main computer controls the 3D printing device to print the 3D model, producing the prosthetic body;
The main computer controls the film-making device to manufacture the surface film for the missing area;
The main computer controls the 2D printing device to print the 2D unfolded image onto the surface film of the missing area, forming the skin.
The main computer controls the assembly equipment to bond the skin to the surface of the prosthesis body. After surface treatment, the prepared prosthesis is obtained.

2. A fully computer-intelligent method for producing high-similarity prostheses as claimed in claim 1, characterized in that, when the healthy reference side lacks symmetry at the missing site, the main computer acquires patient information and utilizes a pre-built artificial intelligence model to generate a 3D model containing segmentation information of each tissue within the patient's missing site. Based on the acquired patient information and the 3D model, a 2D unfolded image of the 3D model's surface layer is obtained, comprising: and the 3D model, it generates a 2D unfolded image of the 3D model's surface layer, including:
The main computer utilizes a pre-built first model reconstruction neural network to perform three-dimensional reconstruction of the missing region by collecting information from the patient's remaining areas. This yields a reconstructed three-dimensional structural model and generates a surface image of the reconstructed missing region. The first model reconstruction neural network is implemented using generative AI technology.
The three-dimensional structural model undergoes internal tissue segmentation using a pre-built second structural hierarchical neural network.
Model refinement is performed using model attribute information from the collected patient data and a pre-built third model modification neural network, yielding a processed 3D model.
Based on the 3D model, the reconstructed surface image of the missing area undergoes two-dimensional image unfolding to produce a 2D unfolded image.

3. The fully computer-intelligent high-similarity prosthesis production method according to claim 1, characterized in that, when a symmetrical healthy reference side exists for the missing area, the main computer acquires patient information, utilizes a pre-built artificial intelligence model to obtain a 3D model containing segmentation information of each tissue within the patient's missing area, and based on the acquired patient and the 3D model, a 2D unfolded image corresponding to the surface layer of the 3D model is obtained, including:
The main computer acquires depth information of the healthy reference side and the remaining portion of the missing area using a depth information acquisition device, and acquires multiple surface images of the healthy reference side at different angles using an image acquisition device;
Perform three-dimensional reconstruction of the missing region using the acquired depth information to obtain the reconstructed three-dimensional structural model;
Perform structural segmentation of the internal organization of the three-dimensional structural model using a pre-built second structural hierarchical neural network, and modify the model using model attribute information from the acquired patient data and a pre-built third model modification neural network, yielding a processed 3D model;
Perform two-dimensional image unfolding of the acquired multiple surface images based on the 3D model, obtaining 2D unfolded images.

4. A fully computer-intelligent high-similarity prosthesis production method according to claim 2, characterized in that the model attribute-related information includes: the patient's age, gender, height, weight, and skin tone;
the model modifications include modifying the prosthesis size, shape, surface color, and surface texture.

5. A fully computer-intelligent high-similarity prosthesis production method according to claim 3, characterized in that the model attribute-related information includes: the patient's age, gender, height, weight, and skin tone;
the model modifications include modifying the prosthesis size, shape, surface color, and surface texture.

6. A fully computer-intelligent high-similarity prosthesis production method according to claim 2, characterized in that the process of obtaining the 2D unfolded image employs a preset inverse filtering algorithm. This algorithm compares the digital image of the 3D model's surface layer with its printed image obtained via a 2D printing device, compensating for the visual discrepancy between the digital image and its printed counterpart to compensate for differences in their actual visual appearance, thereby obtaining the 2D unfolded image for printing. The digital image includes: When the missing part lacks a symmetrical healthy reference side, the 2D unfolded image of the surface reconstructed for the missing part based on the 3D model; or, when a symmetrical healthy reference side exists for the missing area, the two-dimensional unfolded image derived from multiple collected surface images based on the 3D model.

7. A fully computer-intelligent high-similarity prosthesis production method according to claim 3, characterized in that the process of obtaining the 2D unfolded image employs a preset inverse filtering algorithm. This algorithm compares the digital image of the 3D model's surface layer with its printed image obtained via a 2D printing device, compensating for the visual discrepancy between the digital image and its printed counterpart to compensate for differences in their actual visual appearance, thereby obtaining the 2D unfolded image for printing. The digital image includes: When the missing part lacks a symmetrical healthy reference side, the 2D unfolded image of the surface reconstructed for the missing part based on the 3D model; or, when a symmetrical healthy reference side exists for the missing area, the two-dimensional unfolded image derived from multiple collected surface images based on the 3D model.

8. A fully computer-intelligent method for producing high-similarity holiday bodies as claimed in claim 6, characterized in that the process of the preset reverse filtering algorithm comprises:
Step c1, reading initial parameters; wherein said initial parameters include image matrix size, similarity threshold, and maximum iteration count;
Step c2, acquire the digital image;
Step c3: Print the digital image using a 2D printing device to obtain the original printed image;
Step c4: Acquire images of the digital image and the original printed image using the same image acquisition device employed in obtaining the digital image, yielding corresponding acquired images;
Step c5: Calculate the inverse filtering function using the acquired images corresponding to the current digital image and the original printed image;
Step c6: Apply the inverse filtering function to the current digital image to obtain the inverse-filtered digital image;
Step c7: Determine whether the current iteration count is less than the maximum iteration count; if so, proceed to step c8; if not, proceed to step c14.
Step c8: Print the current reverse-filtered digital image using a 2D printing device to obtain the first printed image;
Step c9: Using the same image acquisition device, capture images of both the current reverse-filtered digital image and the first printed image to obtain corresponding captured images;
Step c10: Calculate the similarity index for the captured images corresponding to the currently obtained reverse-filtered digital image and the first printed image;
Step c11: Determine whether the similarity index exceeds the similarity threshold; if yes, proceed to step c12; if no, proceed to step c13;
Step c12: Determine that the currently obtained reverse-filtered digital image serves as the 2D unfolded image to be printed;
Step c13: Store the currently obtained reverse-filtered digital image and its similarity index. Replace the current digital image with the currently obtained reverse-filtered digital image. Replace the capture image corresponding to the current digital image with the capture image corresponding to the currently obtained reverse-filtered digital image. Replace the capture image corresponding to the current original printed image with the capture image corresponding to the current first printed image.
Step c14: Locate the digitally filtered image with the highest similarity index among all iterations and designate it as the 2D unfolded image for printing.

9. A fully computer-intelligent method for producing high-similarity holiday bodies as claimed in claim 7, characterized in that the process of the preset reverse filtering algorithm comprises:
Step c1, reading initial parameters; wherein said initial parameters include image matrix size, similarity threshold, and maximum iteration count;
Step c2, acquire the digital image;
Step c3: Print the digital image using a 2D printing device to obtain the original printed image;
Step c4: Acquire images of the digital image and the original printed image using the same image acquisition device employed in obtaining the digital image, yielding corresponding acquired images;
Step c5: Calculate the inverse filtering function using the acquired images corresponding to the current digital image and the original printed image;
Step c6: Apply the inverse filtering function to the current digital image to obtain the inverse-filtered digital image;
Step c7: Determine whether the current iteration count is less than the maximum iteration count; if so, proceed to step c8; if not, proceed to step c14.
Step c8: Print the current reverse-filtered digital image using a 2D printing device to obtain the first printed image;
Step c9: Using the same image acquisition device, capture images of both the current reverse-filtered digital image and the first printed image to obtain corresponding captured images;
Step c10: Calculate the similarity index for the captured images corresponding to the currently obtained reverse-filtered digital image and the first printed image;
Step c11: Determine whether the similarity index exceeds the similarity threshold; if yes, proceed to step c12; if no, proceed to step c13;
Step c12: Determine that the currently obtained reverse-filtered digital image serves as the 2D unfolded image to be printed;
Step c13: Store the currently obtained reverse-filtered digital image and its similarity index. Replace the current digital image with the currently obtained reverse-filtered digital image. Replace the capture image corresponding to the current digital image with the capture image corresponding to the currently obtained reverse-filtered digital image. Replace the capture image corresponding to the current original printed image with the capture image corresponding to the current first printed image.
Step c14: Locate the digitally filtered image with the highest similarity index among all iterations and designate it as the 2D unfolded image for printing.

10. The fully computer-intelligent high-similarity prosthesis production method according to claim 1, characterized in that the main computer controls the 3D printing device to print the 3D model, obtaining the prosthesis body, comprising:
the main computer controlling the 3D printing device to print using materials matched to each tissue in the 3D model, obtaining the prosthesis body; wherein the materials matched to each tissue comprise multiple optional materials.

11. The fully computer-intelligent high-similarity prosthesis production method according to claim 10, characterized in that for each tissue type, the matched material is determined from the multiple optional materials for that tissue based on collected patient information, selecting one that most closely matches the patient's actual human tissue in terms of hardness and viscoelasticity.

12. The fully computer-intelligent high-similarity prosthesis production method according to claim 1, characterized in that, prior to the main computer controlling the 2D printing device to print the 2D unfolded image onto the surface film of the missing area to form the skin covering, the method further comprises:
The main computer controls the printing system to treat the surface film of the missing area with a surface treatment solution, and uniformly prints pigment ink after treatment.

13. A fully computer-intelligent high-similarity prosthesis production method according to claim 1, characterized in that the main computer controls the assembly equipment to bond the skin to the surface of the prosthesis body, comprising: the main computer controlling the printing system to apply adhesive and lubricant to the surface of the prosthesis body, covering the surface of the coated prosthesis body with the skin, and controlling the uniform pressure instrument to perform pressure treatment, causing the skin and the prosthesis body to tightly adhere and form an integral structure.

14. The fully computer-intelligent high-fidelity prosthesis production method according to claim 13, characterized in that: the density and adhesive strength of the glue match the collected patient information; the lubricant employs a bio-compatible lubricant.

15. The fully computer-intelligent high-similarity prosthesis production method according to claim 1, characterized in that the surface treatment includes: the main computer controlling the spray painting system to coat the surface of the prosthesis body after skin attachment with a protective layer similar to human skin surface.

16. A fully computer-intelligent high-fidelity prosthesis production system, characterized in that it comprises: a main computer and other components; wherein the main computer controls the other components to perform any method step described in claim 1; and wherein the other components include an image acquisition and storage system, film-making equipment, assembly equipment, printing systems, uniform pressure application instruments, and spray coating systems, wherein the image acquisition and storage system comprises depth information acquisition equipment and image acquisition equipment; wherein the printing system comprises at least 3D printing equipment and 2D printing equipment.

17. A fully computer-intelligent high-fidelity prosthesis production system, characterized in that it comprises: a main computer and other components; wherein the main computer controls the other components to perform any method step described in claim 2; and wherein the other components include an image acquisition and storage system, film-making equipment, assembly equipment, printing systems, uniform pressure application instruments, and spray coating systems, wherein the image acquisition and storage system comprises depth information acquisition equipment and image acquisition equipment; wherein the printing system comprises at least 3D printing equipment and 2D printing equipment.

18. A fully computer-intelligent high-fidelity prosthesis production system, characterized in that it comprises: a main computer and other components; wherein the main computer controls the other components to perform any method step described in claim 3; and wherein the other components include an image acquisition and storage system, film-making equipment, assembly equipment, printing systems, uniform pressure application instruments, and spray coating systems, wherein the image acquisition and storage system comprises depth information acquisition equipment and image acquisition equipment; wherein the printing system comprises at least 3D printing equipment and 2D printing equipment.

19. A fully computer-intelligent high-fidelity prosthesis production system, characterized in that it comprises: a main computer and other components; wherein the main computer controls the other components to perform any method step described in claim 4; and wherein the other components include an image acquisition and storage system, film-making equipment, assembly equipment, printing systems, uniform pressure application instruments, and spray coating systems, wherein the image acquisition and storage system comprises depth information acquisition equipment and image acquisition equipment; wherein the printing system comprises at least 3D printing equipment and 2D printing equipment.

20. A fully computer-intelligent high-fidelity prosthesis production system, characterized in that it comprises: a main computer and other components; wherein the main computer controls the other components to perform any method step described in claim 5; and wherein the other components include an image acquisition and storage system, film-making equipment, assembly equipment, printing systems, uniform pressure application instruments, and spray coating systems, wherein the image acquisition and storage system comprises depth information acquisition equipment and image acquisition equipment; wherein the printing system comprises at least 3D printing equipment and 2D printing equipment.

21. A fully computer-intelligent high-fidelity prosthesis production system, characterized in that it comprises: a main computer and other components; wherein the main computer controls the other components to perform any method step described in claim 6; and wherein the other components include an image acquisition and storage system, film-making equipment, assembly equipment, printing systems, uniform pressure application instruments, and spray coating systems, wherein the image acquisition and storage system comprises depth information acquisition equipment and image acquisition equipment; wherein the printing system comprises at least 3D printing equipment and 2D printing equipment.

22. A fully computer-intelligent high-fidelity prosthesis production system, characterized in that it comprises: a main computer and other components; wherein the main computer controls the other components to perform any method step described in claim 7; and wherein the other components include an image acquisition and storage system, film-making equipment, assembly equipment, printing systems, uniform pressure application instruments, and spray coating systems, wherein the image acquisition and storage system comprises depth information acquisition equipment and image acquisition equipment; wherein the printing system comprises at least 3D printing equipment and 2D printing equipment.

23. A fully computer-intelligent high-fidelity prosthesis production system, characterized in that it comprises: a main computer and other components; wherein the main computer controls the other components to perform any method step described in claim 8; and wherein the other components include an image acquisition and storage system, film-making equipment, assembly equipment, printing systems, uniform pressure application instruments, and spray coating systems, wherein the image acquisition and storage system comprises depth information acquisition equipment and image acquisition equipment; wherein the printing system comprises at least 3D printing equipment and 2D printing equipment.

24. A fully computer-intelligent high-fidelity prosthesis production system, characterized in that it comprises: a main computer and other components; wherein the main computer controls the other components to perform any method step described in claim 9; and wherein the other components include an image acquisition and storage system, film-making equipment, assembly equipment, printing systems, uniform pressure application instruments, and spray coating systems, wherein the image acquisition and storage system comprises depth information acquisition equipment and image acquisition equipment; wherein the printing system comprises at least 3D printing equipment and 2D printing equipment.

25. A fully computer-intelligent high-fidelity prosthesis production system, characterized in that it comprises: a main computer and other components; wherein the main computer controls the other components to perform any method step described in claim 10; and wherein the other components include an image acquisition and storage system, film-making equipment, assembly equipment, printing systems, uniform pressure application instruments, and spray coating systems, wherein the image acquisition and storage system comprises depth information acquisition equipment and image acquisition equipment; wherein the printing system comprises at least 3D printing equipment and 2D printing equipment.

26. A fully computer-intelligent high-fidelity prosthesis production system, characterized in that it comprises: a main computer and other components; wherein the main computer controls the other components to perform any method step described in claim 11; and wherein the other components include an image acquisition and storage system, film-making equipment, assembly equipment, printing systems, uniform pressure application instruments, and spray coating systems, wherein the image acquisition and storage system comprises depth information acquisition equipment and image acquisition equipment; wherein the printing system comprises at least 3D printing equipment and 2D printing equipment.

27. A fully computer-intelligent high-fidelity prosthesis production system, characterized in that it comprises: a main computer and other components; wherein the main computer controls the other components to perform any method step described in claim 12; and wherein the other components include an image acquisition and system, film-making storage equipment, assembly equipment, printing systems, uniform pressure application instruments, and spray coating systems, wherein the image acquisition and storage system comprises depth information acquisition equipment and image acquisition equipment; wherein the printing system comprises at least 3D printing equipment and 2D printing equipment.

28. A fully computer-intelligent high-fidelity prosthesis production system, characterized in that it comprises: a main computer and other components; wherein the main computer controls the other components to perform any method step described in claim 13; and wherein the other components include an image acquisition and storage system, film-making equipment, assembly equipment, printing systems, uniform pressure application instruments, and spray coating systems, wherein the image acquisition and storage system comprises depth information acquisition equipment and image acquisition equipment; wherein the printing system comprises at least 3D printing equipment and 2D printing equipment.

29. A fully computer-intelligent high-fidelity prosthesis production system, characterized in that it comprises: a main computer and other components; wherein the main computer controls the other components to perform any method step described in claim 14; and wherein the other components include an image acquisition and storage system, film-making equipment, assembly equipment, printing systems, uniform pressure application instruments, and spray coating systems, wherein the image acquisition and storage system comprises depth information acquisition equipment and image acquisition equipment; wherein the printing system comprises at least 3D printing equipment and 2D printing equipment.

30. A fully computer-intelligent high-fidelity prosthesis production system, characterized in that it comprises: a main computer and other components; wherein the main computer controls the other components to perform any method step described in claim 15; and wherein the other components include an image acquisition and storage system, film-making equipment, assembly equipment, printing systems, uniform pressure application instruments, and spray coating systems, wherein the image acquisition and storage system comprises depth information acquisition equipment and image acquisition equipment; wherein the printing system comprises at least 3D printing equipment and 2D printing equipment.

31. A computer-readable storage medium, characterized in that the computer-readable storage medium stores a computer program, wherein when executed by a processor, the computer program implements the method steps described in claim 1.

32. A computer-readable storage medium, characterized in that the computer-readable storage medium stores a computer program, wherein when executed by a processor, the computer program implements the method steps described in claim 2.

33. A computer-readable storage medium, characterized in that the computer-readable storage medium stores a computer program, wherein when executed by a processor, the computer program implements the method steps described in claim 3.

34. A computer-readable storage medium, characterized in that the computer-readable storage medium stores a computer program, wherein when executed by a processor, the computer program implements the method steps described in claim 4.

35. A computer-readable storage medium, characterized in that the computer-readable storage medium stores a computer program, wherein when executed by a processor, the computer program implements the method steps described in claim 5.

36. A computer-readable storage medium, characterized in that the computer-readable storage medium stores a computer program, wherein when executed by a processor, the computer program implements the method steps described in claim 6.

37. A computer-readable storage medium, characterized in that the computer-readable storage medium stores a computer program, wherein when executed by a processor, the computer program implements the method steps described in claim 7.

38. A computer-readable storage medium, characterized in that the computer-readable storage medium stores a computer program, wherein when executed by a processor, the computer program implements the method steps described in claim 8.

39. A computer-readable storage medium, characterized in that the computer-readable storage medium stores a computer program, wherein when executed by a processor, the computer program implements the method steps described in claim 9.

40. A computer-readable storage medium, characterized in that the computer-readable storage medium stores a computer program, wherein when executed by a processor, the computer program implements the method steps described in claim 10.

41. A computer-readable storage medium, characterized in that the computer-readable storage medium stores a computer program, wherein when executed by a processor, the computer program implements the method steps described in claim 11.

42. A computer-readable storage medium, characterized in that the computer-readable storage medium stores a computer program, wherein when executed by a processor, the computer program implements the method steps described in claim 12.

43. A computer-readable storage medium, characterized in that the computer-readable storage medium stores a computer program, wherein when executed by a processor, the computer program implements the method steps described in claim 13.

44. A computer-readable storage medium, characterized in that the computer-readable storage medium stores a computer program, wherein when executed by a processor, the computer program implements the method steps described in claim 14.

45. A computer-readable storage medium, characterized in that the computer-readable storage medium stores a computer program, wherein when executed by a processor, the computer program implements the method steps described in claim 15.
